(19) United States
(12) Patent Application Publication
(10) Pub. No.: US 2023/0190163 A1
(43) Pub. Date: Jun. 22, 2023

(54) WEARABLE MEDICAL DEVICE SYSTEM WITH CONDUCTIVE INK

(71) Applicant: West Affum Holdings Corp., Grand Cayman (KY)

(72) Inventors: Brian J. Bennett, Redmond, WA (US); Joseph L. Sullivan, Kirkland, WA (US)

(21) Appl. No.: 18/078,850

(22) Filed: Dec. 9, 2022

Related U.S. Application Data
(60) Provisional application No. 63/291,890, filed on Dec. 20, 2021.

(51) Int. Cl. A61B 5/263; A61B 5/318; A61N 1/39

(52) U.S. Cl. CPC A61B 5/263; A61B 5/318; A61N 1/3904

(57) ABSTRACT

Technologies and implementations related to utilizing conductive ink with medical device systems to facilitate electrical contact between an electrode and a person’s skin. The conductive ink may be applied in a variety of manners such as, but not limited to, permanent, semi-permanent, and/or temporary.

### RELATED APPLICATION

This application claims benefit of priority to U.S. Provisional Pat. Application Serial number 63/291,890, filed on Dec. 20, 2021, titled WEARABLE MEDICAL DEVICE SYSTEM WITH CONDUCTIVE INK, which is incorporated herein by reference in its entirety for all purposes.

### INFORMATION

Technology has contributed to improvements in healthcare. Some examples of the application of healthcare related technology may include medical devices that may facilitate monitoring of various health related activities of a person. For example, a medical device may be utilized to monitor the heart activity of a person. The heart activity monitored by the medical device may be in the form of electrical signals (i.e., electrocardiogram or ECG). Monitoring of a person’s ECG may facilitate treatment and/or intervention of heart related issues.

As example of a medical device that may be used to monitor and facilitate treatment and/or intervention of a person’s heart activity may be a cardioverter defibrillator type medical device. In order to help facilitate monitoring and treatment/intervention, a medical device may include components that may be configured to adhere on the body of the person (i.e., adhere on the skin of the person). Some examples of components that may be configured to adhere on the body of the person may include electrodes.

Some electrodes may be utilized to detect electrical signals from the heart activity of the person. For example, electrodes may be utilized to detect ECG signals from the heart to be processed by a medical device. Electrodes utilized to detect electrical signals may be referred to as monitor electrodes.

Some electrodes may be utilized to provide treatment/therapy to the heart of the person. For example, electrodes may be utilized to provide an electric shock to the heart of the person during some form of heart related episode. Electrodes that may be utilized to shock the heart of the person may be included in a defibrillator type medical device. Some electrodes may be utilized to provide a periodic relative smaller electric shocks to provide some form of pacing of the heart of the person. Electrodes utilized to provide electric shocks of some form may be referred to as therapy electrodes.

The monitoring and/or therapy of the heart of the person (i.e., receiving and/or transmitting electrical signals / ECG and/or shock) through the skin may be affected by the transmission of the electrical signals between the skin and the electrode (i.e., a skin/electrode interface).

All subject matter discussed in this Information section of this document is not necessarily prior art and may not be presumed to be prior art simply because it is presented in this Information section. Plus, any reference to any prior art in this description is not, and should not be taken as, an acknowledgement or any form of suggestion that such prior art forms parts of the common general knowledge in any art in any country. Along these lines, any recognition of problems in the prior art discussed in this Information section or associated with such subject matter should not be treated as prior art, unless expressly stated to be prior art. Rather, the discussion of any subject matter in this Information section should be treated as part of the approach taken towards the particular problem by the inventor(s). This approach in and of itself may also be inventive.

### SUMMARY

Described herein are various illustrative systems and methods for improved electrical contact between a person’s (patient’s) skin and an electrode of a medical device system. Some example medical device systems may include an electrode adapted to contact the patient’s skin to sense electrical signals in a patient’s body, while the patient is using the medical device system. Some example medical device systems may include a portion of conductive ink adapted to be applied to a patient’s skin at a location at which the electrode contacts the patient’s skin while the patient is using the medical device system.

Some example methods may include using conductive ink in a wearable medical device system. The example method may include applying a portion of conductive ink to the patient’s skin at a location at which an electrode is configured to contact the skin while the patient is wearing the wearable medical device. The example method may include contacting the electrode to the skin at the location at which the portion of the conductive ink was applied, sensing electrical signals of the patient’s body via the electrode while the electrode is contacting the skin, and monitoring the patients’ electrical signals via the electrode.

Some example methods may include using conductive ink in a wearable medical device system. The example method may include applying a portion of conductive ink to the patient’s skin at a location at which a therapy electrode is configured to contact the skin while the patient is wearing the wearable medical device. The example method may also include contacting the therapy electrode to the skin at the location at which the portion of the conductive ink was applied and discharging electrical therapy via the therapy electrode while the therapy electrode is contacting the skin.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIGS. 1A and 1B illustrate a front and back view of conductive ink disposed on a person, in accordance with various embodiments.

FIG. 2 illustrates a wearable medical device (WMD), which may be utilized with various embodiments.

FIG. 3 is a block diagram illustrating components of a medical device, which may be used with various embodiments.

### DETAILED DESCRIPTION

The following description sets forth various examples along with specific details to provide a thorough understanding of claimed subject matter. It will be understood by those skilled in the art after review and understanding of the present disclosure, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

The disclosure is drawn, inter alia, to apparatus, and systems related to a providing an improved electrical contact between the skin of a person and an electrode of a medical device.

Medical devices may be utilized to facilitate monitoring and/or treatment of various medical conditions of a person. For example, a medical device may be utilized to facilitate monitoring the activities of the heart of the person. To facilitate the monitoring of the activities of the heart, the medical device may be of a heart monitoring type (e.g., monitoring the electrical activity of the heart). A medical device utilized to monitor the heart may include an ECG monitoring type medical device. The ECG monitoring medical device may include a number of electrodes. The electrodes may be attachable on the skin of the person and may be configured to receive electrical signals from the activities of the heart. The quality of the electrical signals from the skin of the person may be affected by the electrical contact between the electrode and the skin (i.e., electrode-skin interface).

In another example, a medical device may be utilized to facilitate treatment and/or therapy of the heart of the person. In one example, in order to facilitate the treatment/therapy of the heart, the medical device may provide a shock to defibrillate the heart. In another example, in order to facilitate treatment/therapy of the heart, the medical device may provide periodic or as needed shocks to pace the heart. A medical device utilized to monitor the heart may include a defibrillator and/or pacing type medical device. The defibrillator and/or pacing type medical device may include a electrodes to provide the electric shock(s) to the heart. Here again, the quality or strength of the electrical signals to the heart via the skin of the person may be affected by the electrical contact between the electrode and the skin (i.e., electrode-skin interface).

In both cases described above (i.e., ECG and defibrillator and/or pacing), the electrical contact at the electrode-skin interface may affect the electrical signals either received and/or transmitted. Accordingly, in monitoring, external defibrillating, and/or pacing of a person’s heart, electrodes may be configured to contact the skin (i.e., electrode-skin interface), and when electrical activity is active, may create an electrical path from the defibrillator through the person and back to the defibrillator. A contact impedance between the electrode and skin (electrode-skin interface) may affect the quality of the monitored information, and in case of energy delivery, may cause a reduction in electrical energy and/or current bunching/hot-spots. Hot-spots may cause unnecessary discomfort for the patient, which may include burns. Some methodologies may include the application of a conductive gel at the electrode-skin interface, and/or the utilization of an adhesive, stick-on electrodes, which may be expensive, messy, cumbersome, and/or bulky.

Before turning to the Figures, some non-limiting example scenarios of utilization of the claimed subject matter may be described. In one non-limiting example scenario, a person’s heart (i.e., hereon, a patient’s heart) may be monitored by a medical device. The medical device may be configured to monitor the activity of the patient’s heart by receiving electrical signals from the patient’s heart (i.e., ECG signal). To facilitate the receiving of the ECG signal, the medical device may include a number of monitor electrodes. The monitor electrodes may be adapted to contact the patient’s skin to receive (i.e., sense) electrical signals in the patient’s body (e.g., the patient’s heart). In accordance with various embodiments, the electrode-skin interface may be positively affected.

Some examples of positively affecting the electrode-skin interface may include reduction of negative electrical properties at the electrode-skin interface. Negative electrical properties may include skin impedance.

Continuing with the scenario, in one example, conductive ink may be utilized to positively affect the electrode-skin interface. For example, a reduction of skin impedance may be facilitated by utilizing conductive ink. A portion of the conductive ink may be adapted to be applied to the patient’s skin at or substantially close to the skin area or areas, where an electrode or electrodes are to be placed.

The application of the conductive ink may be achieved in a variety of methods. In one example, conductive ink may be applied to the patient’s skin as a tattoo. For example, a conductive skin tattoo may be helpful, when a person is to wear the electrodes for an extended period of time.

Continuing with the scenario, in one example, the medical device may have been prescribed for the patient (i.e., by prescription) for a period of time. Accordingly, the conductive ink may be temporary, where temporary conductive tattoo(s) may be formulated to last for the expected duration of the prescription or reapplied as the temporary tattoo(s) fades.

In various examples, the conductive ink may be applied utilizing a wide variety of methods such as, but not limited to, via a tattoo, decalcomania, brush, or other types of ink dispensers.

In some examples, the conductive ink may be applied to areas of the patient’s skin, where the electrodes are to be positioned. Continuing with the scenario, the applied conductive ink may help the patient and/or the patient’s caregiver to position the electrodes more accurately. For example, if the patient takes the medical device system off to shower, the patient may use the conductive markings as a guide for positioning the electrodes, when the patient puts the medical device system back on. Accordingly, in some example embodiments, the conductive ink area may be larger than the electrode area. The conductive ink area being larger than the electrode may facilitate a large adjustment in electrode location.

In some example embodiments, the conductive ink area/size and/or color for different electrodes may be different (e.g., ink area/size and/or color for monitor electrodes and/or therapy electrodes may be different). The conductive ink area/size and/or color being different may further facilitate a proper placement of the electrodes on the patient’s skin (e.g., proper locations for the monitor electrodes and/or for the therapy electrodes).

In some example embodiments, the conductive material may be of a conductive polymer type that may be applied to the patient’s skin by painting the conductive polymer onto the patient’s skin. For example, the painted conductive polymer on the patient’s skin may be adapted to adhere to the skin even when the electrodes are removed and then placed back on the patient’s skin. Accordingly, the conductive paint may facilitate an improved contact with the electrodes, while reducing the impedance, and may further assist in more accurately positioning the electrode on the patient’s skin during removal and placement.

As may be appreciated, the conductive ink may be utilized in a variety of manners in/on the patient’s skin. For example, the conductive ink can be painted on the skin, without the need to inject it into the skin as may be the case in tattoo application. However, in some example embodiments, a combination of both the application of the conductive ink on the surface of the patient’s skin (e.g., painted) and the application of the conductive ink under the surface of the patient’s skin (e.g., tattooed) may be utilized.

As may be appreciated from the above description, in some example embodiments, the conductive material may be of a conductive paint and/or of a polymer painted type disposed onto an area indicating where an electrode is to be positioned. In some example embodiments, a combination of conductive materials such as, but not limited to, conductive ink and/or polymer may be utilized.

In some example embodiments, of the medical device system may be wearable such as, but not limited to, a wearable medical device (WMD) having electrodes, where a support structure such as, but not limited to, a garment may facilitate proper contact of the electrodes against the patient’s skin. As previously mentioned, the conductive ink may reduce the impedance and/or a barrier to reach the patient’s heart due to positively affecting the electrical connection between the medical device system and patient’s skin.

It may be appreciated that permanent tattoos may be disposed in the dermis layer of a person’s skin and may stay in the dermis layer for an extended period of time (i.e., permanent tattoo). However, if the ink is disposed in the epidermis layer of a person’s skin, the ink may be temporary, which may still be sufficient (i.e., for a short period of time such as the prescription period). For the ink disposed in the epidermis layer, a follow-up tattoo may be applied to maintain epidermal conduction for longer prescriptions.

For purposes of a WMD, a permanent tattoo may be utilized to facilitate reduction of the overall patient’s impedance. Alternatively, a temporary tattoo may be utilized to apply conductive ink to the patient’s skin. The application of the temporary tattoo may be similar to a permanent tattoo (e.g., use of a needle). However, in the case of the temporary tattoo, the needle may be utilized to limit the penetration into the patient’s skin (i.e., disposing in the epidermis layer). In another example, the needle associated with common tattooing techniques may not be utilized, and instead, an alternative method may be utilized.

In some example embodiments, a temporary tattoo of conductive material such as ink may be applied onto the patient’s skin. In one example, a temporary tattoo may be applied utilizing transfer paper.

In another example, a conductive ink paint, that may last on the skin for a period of time, may be applied onto the epidermis of the patient’s skin. For example, the conductive ink may be wetted and held on the skin long enough for the ink to adhere/transfer to the skin.

For some temporary tattoo embodiments, (i.e., in which conductive ink may be applied to the skin without utilizing a tattooing needle), a take-home temporary tattoo may be provided to the patient to take home with them. The take-home temporary tattoo may be adapted to facilitate the patient in reapplying the temporary tattoo periodically (e.g., every couple weeks or as often as is determined necessary for the specific type of temporary tattoo). Similarly, for some temporary conductive ink painting embodiments, additional conductive ink may be provided to the patient for reapplication.

As previously described, in some alternative example embodiments, painting conductive ink on the patient’s skin can be utilized. Painting the conductive ink on the patient’s skin may help facilitate ease with which the patient may perform self-maintenance. For example, as the areas of the conductive ink that has been painted onto the patient’s skin fades, peels off, and/or is rubbed off, the patient may be provided the opportunity to touch-up these areas with conductive ink provided to the patient.

In the above descriptions/scenarios, monitor electrodes may have been described. However, it should be appreciated that the description may be applicable to therapy electrodes. For example, the medical device may be a WMD, where the WMD may be a wearable cardioverter defibrillator (WCD) device. Accordingly, the claimed subject matter is not limited in this respect.

In the example of the WCD device, the WCD may be configured to facilitate monitoring and treatment of potential issues with a person’s heart (i.e., patient’s heart). For example, a patient may have a health condition, where the patient may require a defibrillator (e.g., Arrhythmia). The defibrillator may be in the form of a cardioverter defibrillator (WCD), which may help facilitate prevention of Sudden Cardiac Death (SCD). The WCD may be wearable by the patient (e.g., of a garment type). The WCD may include a variety of electrodes (e.g., monitor electrodes and therapy electrodes). The electrodes of the WCD may be configured to contact the patient’s skin using the various methods as described herein. Having the improved conductivity at the electrode-skin interface may facilitate detection of the heartbeat issue, and responsive to the detected heart best issue, the WCD module may facilitate treatment of the person’s heart via the electrodes.

Even though some of the disclosure may be described with respect to conductive ink, it is contemplated within the scope of the disclosure the conductive ink may include material that may further help facilitate placement of an electrode on a patient’s skin. For example, a conductive ink may include material that may facilitate a magnetic force. The conductive ink may include metallic material to facilitate adhesion with an electrode magnetically. The metallic material may be material such as, but not limited to, very small metallic particles included in the conductive ink (e.g., iron, cobalt, steel, nickel, etc. having sizes that may facilitate being included in the ink/aqueous ). In this example, the conductive ink may be painted on the patient’s skin. The electrode may include a magnetic material as well. When the electrode is placed on the location of the conductive ink, the adhesion may be facilitated by the magnetic force. The magnetic force may help to facilitate reduction in issues with adhesion/contact between the electrode and the patient’s skin.

The conductive ink may include non-metallic material based magnets such as, but not limited to, graphene based material. Additionally, the conductive ink may include rare-earth materials such as, but not limited to, lanthanides.

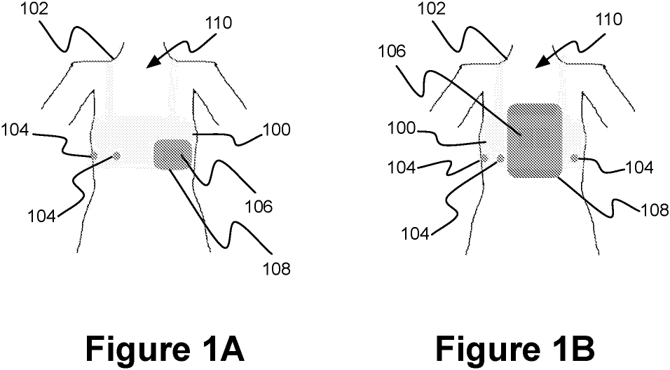

Turning now to FIGS. 1A and 1B, FIGS. 1A and 1B illustrate a front and back view of conductive ink disposed on a person, in accordance with various embodiments. In FIGS. 1A and 1B, a medical device system 100 may be utilized by a person (patient 102). The patient 102 may be shown as wearing the medical device system 100 (e.g., a WMD and/or a WCD). The medical device system 100 may include a number of monitor electrodes 104 and a couple of therapy electrodes 106.

In FIG. 1A, a front view of the patient 102 may be shown. Shown in the front view of FIG. 1A, a conductive ink 108 may be applied to the patient’s skin 110. As shown in FIG. 1A, a portion of the conductive ink 108 may have been applied at a location at which the therapy electrode 106 may be configured to contact the patient’s skin 110, while the patient 102 is wearing the medical device system 100. In FIG. 1A, the conductive ink 108 may be shown to cover an area similar to the therapy electrode 106. As a result, if the medical device system 100 detects an issue with the patient’s heart (not shown), the medical device system 100 may be adapted to discharge electrical therapy via the therapy electrode 106, while the therapy electrode 106 is in contact with the patient’s skin 110. In accordance with various embodiments, the conductive ink 108 may facilitate improved electrical contact between the therapy electrode 106 and the patient’s skin 110. As a result, electrical impedance may be reduced.

In FIG. 1B, a back view of the patient 102 may be shown. Shown in the back view of FIG. 1B, the conductive ink 108 may be applied to the patient’s skin 110. As shown in FIG. 1B, a portion of the conductive ink 108 may have been applied at a location at which the therapy electrode 106 may be configured to contact the patient’s skin 110, while the patient 102 is wearing the medical device system 100. However, in FIG. 1B, the conductive ink 108 may be shown to cover an area much larger than the therapy electrode 106. The medical device system 100 may be configured to detect an issue with the patient’s heart (not shown). If the medical device system 100 detects an issue, the medical device system 100 may be adapted to discharge electrical therapy via the therapy electrode 106, while the therapy electrode 106 is in contact with the patient’s skin 110. In accordance with various embodiments, the conductive ink 108 may facilitate improved electrical contact between the therapy electrode 106 and the patient’s skin 110. As a result, electrical impedance may be reduced.

In FIG. 1B, having the area covered by the conductive ink 108 being much larger than the therapy electrode 106 may help to facilitate ease of placement of the therapy electrode 106. For example, because the area covered by the conductive ink 108 is much larger than the therapy electrode 106, it may be easier to place the therapy electrode 106 (larger target), which may be of further assistance being the back of the person.

Even though FIGS. 1A and 1B may show the conductive ink 108 being utilized with the therapy electrodes 106, the conductive ink 108 may be utilized with the monitor electrodes 104 as previously described.

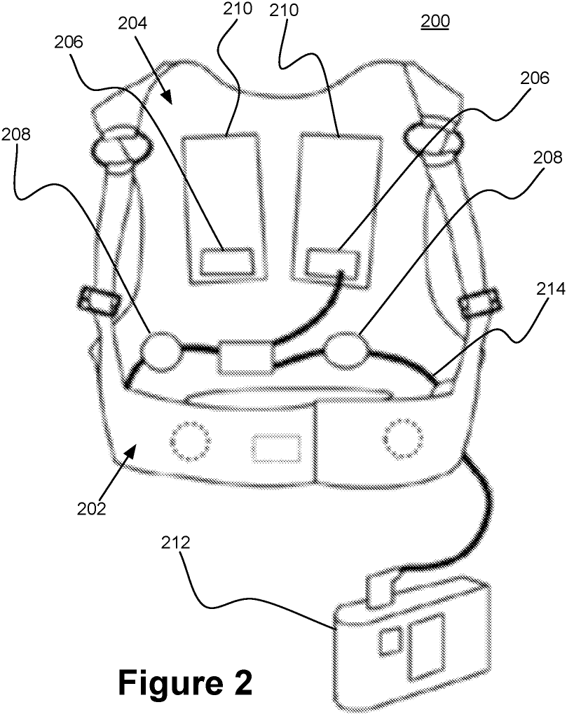

FIG. 2 illustrates a wearable medical device (WMD), which may be utilized with various embodiments. In FIG. 2, a WMD may be configured to facilitate monitoring and treatment of a person’s heart such as, but not limited to, a wearable cardioverter defibrillator (WCD) 200. The WCD 200 may be in the form of a clothing configured to be worn by a user such as, but not limited to, a vest type. Accordingly, the WCD 200 may have a front side 202 and a back side 204 forming the vest type WCD 200 as shown. Additionally, the WCD 200 may include one or more electrodes configured to defibrillate the person’s heart, defibrillator electrodes (therapy electrodes 206) and one or more electrodes configured to detect and measure the person’s electrocardiogram (ECG), monitor electrodes 208.

The WCD 200 may be configured to be worn by the person to facilitate maintenance of the therapy electrodes 206 and the monitor electrodes 208 on the body of the person. For example, therapy electrodes 206 may be included in pockets 210. In one example, the inside of pockets 210 may be made of loose netting to facilitate contact of the therapy electrodes 206 on the back of the person and may include assistance of conductive fluid that may have been deployed for the therapy electrodes 206. Additionally, the monitor electrodes 208 may be maintained in positions that may surround the torso of the person torso, which may facilitate sensing ECG signals and/or the impedance of the person.

In some examples, conductive ink may be utilized to facilitate electrical conductivity with the electrodes as disclosed herein.

It should be appreciated after review of this disclosure that the locations of the therapy electrodes 206 may be shown in various configurations such as, but not limited to, one front and one back, across a chest, across a back, etc. to facilitate defibrillation, and accordingly, the locations of the therapy electrodes 206 and/or the monitor electrodes 208 in FIG. 2 may be for illustrative purposes to show that there may be some electrodes to facilitate operation of the WCD 200. Additionally, for the purposes of the detailed description, references may be made to “an electrode”, which may be any one of electrodes (therapy electrodes 206 and/or monitor electrodes 208) to provide the functionality of the WCD 200.

Continuing to refer FIG. 2, the WCD 200 may include a WCD monitor 212. In the example shown in FIG. 2, the WCD monitor 212 may be communicatively coupled to the therapy electrodes 206 and/or monitor electrodes 208 via one or more wires 214. However, in some other examples, the WCD monitor 212 may be integrated with the WCD such as, but not limited to, the back side 204. The WCD monitor 212 may include various components to facilitate the functionality of the WCD 200 (i.e., monitor and defibrillate the person’s heart) such as, but not limited to, a processor, memory, power supply (e.g., battery), a display, and so forth. Some of these components may be described in further detail later in the disclosure.

It should be appreciated that the example WCD 200 shown in FIG. 2 may be in the form of a vest. However, the WCD 200 may be in the form of a wide variety of clothing such as, but not limited to, a jacket, a t-shirt, a dress shirt, a belt, a blouse, a coat, and any combination thereof. Accordingly, the components of the WCD 200 may be integrated with a wide variety of wearable clothing.

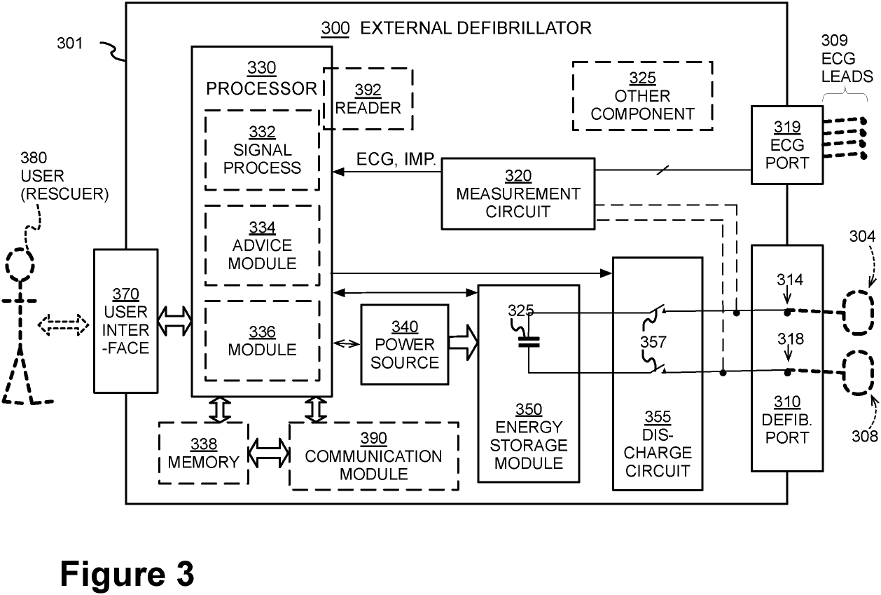

FIG. 3 is a block diagram illustrating components of a medical device (e.g., External Defibrillator 300), which may be used with various embodiments. These components may be, for example, a medical device 100 and 200 (shown in FIGS. 1A, 1B, and 2). For simplicity, the medical device may be an example of a defibrillator device.

The defibrillator device 300 may be intended for use by a user 380 (e.g., the person 102 FIGS. 1A and 1B). The defibrillator device 300 may typically include a defibrillation port 310, such as a socket in housing 301. The defibrillation port 310 may include nodes 314 and 318. One or more electrodes 304 and 308 may be plugged in to the defibrillation port 310, so as to make electrical contact with nodes 314 and 318, respectively. It may also be possible that the electrodes 304 and 308 may be connected continuously to the defibrillation port 310, etc. Either way, the defibrillation port 310 may be used for guiding via the electrodes 304 and 308 to the person 380 an electrical charge that may have been stored in the defibrillator device 300, as described herein.

If the defibrillator device 300 comprise of a heart monitoring component, as was described herein, the defibrillator device 300 may also have an ECG port 319 in the housing 301, for receiving ECG leads 309. The ECG leads 309 may facilitate sensing of an ECG signal (e.g., a 12-lead signal or from a different number of lead signals), and electrode attachment integrity may be determined from the ECG signal, in accordance with the various embodiments disclosed herein. Moreover, a heart monitoring component could have additional ports (not shown), and the other component 325 may be configured to utilize the electrical signal (e.g., ECG signal, impedance, etc. to facilitate determination of electrode leads off from the skin of the user 380), in accordance with various embodiments.

The defibrillator 300 also may include a measurement circuit 320. The measurement circuit 320 may receive physiological signals from the ECG port 319, and also from other ports, if provided (e.g., previously described lead-off circuitry). The circuit 320 may render detected physiological signals and their corresponding information. The information may be in the form of data, or other signals, etc.

The measurement circuit 320 may obtain physiological signals through the nodes 314 and 318 instead, when the electrodes 304 and 308 are attached to the person 380 (i.e., the skin 110 shown in FIGS. 1A and 1B). In these cases, a person’s ECG signal may be detected as a voltage difference between the electrodes 304 and 308. Additionally, the impedance between the electrodes 304 and 308 may detect, among other things, whether the electrodes 304 and 308 have been inadvertently disconnected from the skin of the person 380.

The defibrillator 300 may also include a processor 330. The processor 330 may be implemented in a wide variety of manners for causing actions and operations to be performed. Some examples may include digital and/or analog processors such as microprocessors and digital-signal processors (DSPs), controllers such as microcontrollers, software running in a machine environment, programmable circuits such as Field Programmable Gate Arrays (FPGAs), Field-Programmable Analog Arrays (FPAAs), Programmable Logic Devices (PLDs), Application Specific Integrated Circuits (ASICs), and so on or any combination thereof.

The processor 330 may include a number of modules. One example module may be a signal processing module 332, which may detect outputs from the measurement circuit 320. The signal processing module 332 may include electronic components configured to a establish communication link between the WMD and the server, where the established communication may facilitate capture of portions of ECG data between the WMD and the server as described above. Accordingly, configuration and comparison of portions of ECG data may be facilitated in accordance with one or more embodiments.

In another example, advice module 334 may provide advice based, at least in part, on outputs of signal processing module 332. The advice module 334 may include an algorithm such as, but not limited to, Shock Advisory Algorithm, implement decision rules, and so on. For example, the advice may be to shock, to not shock, to administer other forms of therapy, provide an indication to confirm a health status of the person 380 (e.g., determine whether the person 380 is experiencing perfusing or non-perfusing ventricular tachycardia (VT), and so on. If the advice is to shock, some defibrillator examples may report the advice to the user and prompt them to do it. In other examples, the defibrillator device may execute the advice by administering the shock. If the advice is to administer CPR, the defibrillator 300 may further issue prompts for administrating CPR, and so forth. Examples of Shock Advisory Algorithm may be found in U.S. pat. application USN 15/421,165, filed Jan. 31, 2017 (now issued as U.S. Pat. No. 10,016,614) titled Wearable cardioverter defibrillator (WCD) system making shock/no shock determinations by aggregating aspects of multiple patient parameters, which is incorporated by reference in its entirety for all purposes.

The processor 330 may include additional modules, such as module 336 for various other functions. Additionally, if other component 325 is provided, it may be operated in part by processor 330, etc.

In an example, the defibrillator device 300 may include a memory 338, which may work together with the processor 330. The memory 338 may be implemented in a wide variety of manners. For example, the memory 338 may be implemented such as, but not limited to, nonvolatile memories (NVM), read-only memories (ROM), random access memories (RAM), and so forth or any combination thereof. The memory 338 may include programs for the processor 330, and so on. For example, the memory 338 may include ECG signals for determining a respiration rate post-event. The programs may include operational programs executed by the processor 330 and may also include protocols and methodologies so that decisions may be made by advice module 334. Additionally, the memory 338 may store various prompts for the user 380, etc. Moreover, the memory 338 may store a wide variety of information (i.e., predetermined parameter data) such as, but not limited to information regarding the person 380.

The defibrillator 300 may also include a power source 340. In order to facilitate portability of defibrillator device 300, the power source 340 may include a battery type device. A battery type device may be implemented as a battery pack, which may be rechargeable or not- rechargeable. At times, a combination of rechargeable and non-rechargeable battery packs may be utilized. Examples of power source 340 may include AC power override, where AC power may be available, and so on. In some examples, the processor 330 may control the power source 340.

Additionally, the defibrillator device 300 may include an energy storage module 350. The energy storage module 350 may be configured to store some electrical energy (e.g., when preparing for sudden discharge to administer a shock). The energy storage module 350 may be charged from the power source 340 to an appropriate level of energy, as may be controlled by the processor 330. In some implementations, the energy storage module 350 may include one or more capacitors 352, and the like.

The defibrillator 300 may include a discharge circuit 355. The discharge circuit 355 may be controlled to facilitate discharging of the energy stored in energy storage module 350 to the nodes 314 and 318. The discharge circuit 355 may include one or more switches 357. The one or more switches 357 may be configured in a number of manners such as, but not limited to, an H-bridge, and so forth.

The defibrillator device 300 may further include a user interface 370 for the user 380. The user interface 370 may be implemented in a variety of manners. For example, the user interface 370 may include a display screen capable of displaying what is detected and measured, provide visual feedback to the user 380 for their resuscitation attempts, and so forth. The user interface 370 may also include an audio output such as, but not limited to, a speaker to issue audio prompts, etc. The user interface 370 may additionally include various control devices such as, but not limited to, pushbuttons, keyboards, switches, track pads, and so forth. Additionally, the discharge circuit 355 may be controlled by the processor 330 or directly by the user 380 via the user interface 370, and so forth.

Additionally, the defibrillator device 300 may include other components. For example, a communication module 390 may be provided for transmitting ECG signals stored on the defibrillator device 300 to be downloaded and processed as described above. Such communication may be performed wirelessly, or via wire, or by infrared communication, near field communication (NFC), Bluetooth, WiFi, and so forth. Accordingly, information may be communicated, such as personal data, incident information, therapy attempted, CPR performance, ECG information, and so forth.

A feature of a defibrillator device may be CPR related prompting. CPR prompts may be issued to the user 380 visually or by audio facilitating assistance in the administration of CPR by the user 380. Examples may be found in U.S. Pat. No. 6,334,070 and No. 6,356,785.

It should be appreciated after review of this disclosure that it is contemplated within the scope and spirit of the present disclosure that the claimed subject matter may include a wide variety of healthcare devices. Accordingly, the claimed subject matter is not limited in these respects.

Some portions of the foregoing detailed description are presented in terms of algorithms or symbolic representations of operations on data bits or binary digital signals stored within a computing system memory, such as a computer memory. These algorithmic descriptions or representations are examples of techniques used by those of ordinary skill in the data processing arts to convey the substance of their work to others skilled in the art. An algorithm is here, and generally, considered to be a self-consistent sequence of operations or similar processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals or the like. It should be understood, however, that all of these and similar terms are to be associated with appropriate physical quantities and are merely convenient labels. Unless specifically stated otherwise, as apparent from the following discussion, it is appreciated that throughout this specification discussion utilizing terms such as “processing,” “computing,” “calculating,” “determining” or the like refer to actions or processes of a computing device that manipulates or transforms data represented as physical electronic or magnetic quantities within memories, registers, or other information storage devices, transmission devices, or display devices of the computing device.

Claimed subject matter is not limited in scope to the particular implementations described herein. For example, some implementations may be in hardware, such as those employed to operate on a device or combination of devices, for example, whereas other implementations may be in software and/or firmware. Likewise, although claimed subject matter is not limited in scope in this respect, some implementations may include one or more articles, such as a signal bearing medium, a storage medium and/or storage media. This storage media, such as CD-ROMs, computer disks, flash memory, or the like, for example, may have instructions stored thereon that, when executed by a computing device such as a computing system, computing platform, or other system, for example, may result in execution of a processor in accordance with claimed subject matter, such as one of the implementations previously described, for example. As one possibility, a computing device may include one or more processing units or processors, one or more input/output devices, such as a display, a keyboard and/or a mouse, and one or more memories, such as static random access memory, dynamic random access memory, flash memory, and/or a hard drive.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skilled in the art in light of this disclosure. In addition, those skilled in the art will appreciate that the mechanisms of the subject matter described herein are capable of being distributed as a product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a flexible disk, a hard disk drive (HDD), a Compact Disc (CD), a Digital Versatile Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter is not limited to the particular examples disclosed, but that such claimed subject matter also may include all implementations falling within the scope of the appended claims, and equivalents thereof.

1. A medical device system comprising:
an electrode configured to electrically couple to a patient’s skin, the electrode further configured to sense electrical signals in a patient’s body while the patient is using the medical device system; and
a portion of conductive ink configured to be applied to the patient’s skin at a location proximate to a location electrically coupled between the electrode and the patient’s skin while the patient is using the medical device system.

2. The medical device system of claim 1, wherein the portion of conductive ink comprises conductive ink configured to be applied to the patient’s skin as a tattoo.

3. The medical device system of claim 1, wherein the portion of conductive ink comprises conductive ink configured to be applied to the patient’s skin as a decal.

4. The medical device system of claim 1, wherein the portion of conductive ink comprises conductive ink configured to be applied to the patient’s skin in a paint form.

5. The medical device system of claim 1, wherein the portion of conductive ink comprises two or more of:
conductive ink configured to be applied to the patient’s skin as a tattoo,
conductive ink configured to be applied to the patient’s skin as a decal, or
conductive ink configured to be applied to the patient’s skin in a paint form.

6. The medical device system of claim 1 further comprising a wearable medical device (WMD), the WMD including the electrode.

7. The medical device system of claim 6, wherein the WMD comprises a wearable cardioverter defibrillator (WCD), the WCD including the electrode.

8. A method of using conductive ink in a wearable medical device (WMD) system, the method comprising:
applying a portion of conductive ink to a patient’s skin at a location proximate to an electrode configured to electrically couple to the patient’s skin while the patient is wearing the WMD system;
disposing the electrode proximate to the patient’s skin proximate to at the location where the portion of conductive ink was applied at ;
sensing electrical signals of the patient’s body via the electrode while the electrode is electrically coupled with the skin; and
monitoring the patient’s electrical signals via the electrode.

9. A method of using conductive ink in a wearable medical device (WMD) system, comprising:
applying a portion of conductive ink to a patient’s skin at a location proximate to a therapy electrode configured to electrically couple with the patient’s skin while the patient is wearing the WMD system; and
disposing the therapy electrode proximate to the patient’s skin proximate to at the location where the portion of the conductive ink was applied.

10. The method of claim 8 , wherein the portion of conductive ink comprises conductive ink configured to be applied to the patient’s skin as a tattoo.

11. The method of claim 8 ,wherein the portion of conductive ink comprises conductive ink configured to be applied to the patient’s skin as a decal.

12. The method of claim 8 ,wherein the portion of conductive ink comprises conductive ink configured to be applied to the patient’s skin in a paint form .

13. The method of claim 8 ,wherein the portion of conductive ink comprises two or more of:
conductive ink configured to be applied to the patient’s skin as a tattoo,
conductive ink configured to be applied to the patient’s skin as a decal, or
conductive ink configured to be applied to the patient’s skin in a paint form.

14. The method of claim 8, wherein the WMD comprises a wearable cardioverter defibrillator (WCD).

15. The medical device system of claim 7, wherein the electrode comprises a therapy electrode.

16. The method of claim 9, wherein the portion of conductive ink comprises conductive ink configured to be applied to the patient’s skin as a tattoo.

17. The method of claim 9, wherein the portion of conductive ink comprises conductive ink configured to be applied to the patient’s skin as a decal.

18. The method of claim 9, wherein the portion of conductive ink comprises conductive ink configured to be applied to the patient’s skin in a paint form.

19. The method of claim 9, wherein the portion of conductive ink comprises two or more of:
conductive ink configured to be applied to the patient’s skin as a tattoo,
conductive ink configured to be applied to the patient’s skin as a decal, or
conductive ink configured to be applied to the patient’s skin in a paint form.

20. The method of claim 9, wherein the WMD comprises a wearable cardioverter defibrillator (WCD).
